(19) United States
(12) Patent Application Publication
(10) Pub. No.: US 2007/0239253 A1
(43) Pub. Date: Oct. 11, 2007

(54) Oscillation assisted drug elution apparatus and method

(76) Inventors: Karl Alan Jagger, Deephaven, MN (US); James Lee Shippy III, Maple Grove, MN (US)

Correspondence Address: COOLEY GODWARD KRONISH LLP;ATTN: PATENT GROUP, Suite 500, 1200 - 19th Street, NW, WASHINGTON, DC, 20036-2402, US

(21) Appl. No.: 11/398,670

(22) Filed: Apr. 6, 2006

(51) Int. Cl. A61F 2/84; A61F 2/82

(52) U.S. Cl. 623/1.11; 604/891.1; 623/1.42

(57) ABSTRACT

An apparatus includes a base and a coating disposed on the base. A therapeutic agent is disposed on at least one of the base or the coating. A vibration device is coupled to the base. The vibration device is configured to cause movement of the base such that at least a portion of the therapeutic agent is released from the base or the coating. A method includes inserting a stent into a body lumen of a patient. The stent has a base, a coating disposed on at least a portion of the base, and a therapeutic agent carried by at least one of the base or the coating. The stent is vibrated such that at least a portion of the therapeutic agent is released from the stent.

### BACKGROUND

The invention relates to methods and apparatuses for drug elution within a body lumen of a patient, and more particularly to methods and apparatuses for oscillation assisted drug elution.

Some known intraluminal stents are inserted or implanted into a body lumen, for example, a coronary artery, after a procedure such as percutaneous transluminal coronary angioplasty. Such stents can be used to maintain the patency of a body lumen by supporting the walls of the lumen and preventing abrupt reclosure or collapse thereof. These known stents can also be provided with one or more therapeutic agents adapted to be locally released from the stent at the site of implantation. In the case of a coronary stent, the stent can be adapted to provide release of, for example, an antithrombotic agent to inhibit clotting or an antiproliferative agent to inhibit smooth muscle cell proliferation, i.e., neointimal hyperplasia, which may be a factor leading to re-narrowing or restenosis of the blood vessel after implantation of the stent.

Some known stents are formed from biocompatible metals such as stainless steel, or metal alloys such as nickel-titanium alloys that are often employed because of their desirable shape-memory characteristics. Some known stents are also formed from bioabsorbable metals, such as a magnesium alloy. Metallic materials are advantageously employed to construct stents because of the inherent rigidity of metallic materials and the consequent ability of the metallic stent to maintain patency of the lumen upon implantation of the stent. In addition, some known stents are formed from biocompatible and/or bioabsorbable plastic materials, such as polyethylene terephthalate, polytetrafuoroethylene, polyacetal, poly-L-lactide, polyactic acid, polyanhydrides, polyorthoesters, polyphosphate esters, poly iminocarbonates, polyurethane, polyhydroxy butyrates, polycaprolactones, polytrimethylene carbonates, and biodegradable polymers.

To provide the therapeutic agent, known stents have been coated with a biodegradable or non-biodegradable material containing the therapeutic agent. The coating may also provide a more biocompatible surface directly in contact with the body lumen wall. With such stents, as the bioabsorbable coating is absorbed into the body of the patient, the therapeutic agent is released.

Although stents carrying a therapeutic agent are known, the amount and timing of the release of the therapeutic agent into the body lumen cannot be precisely controlled using known techniques. Drug elution from coated stents can depend, at least in part, on a number of factors including the exposed surface area of the stent, particle diffusion path, coating quality, and coating particle size. Although techniques have been developed to assist in the release of therapeutic agents from a stent, these techniques are mostly mechanical in nature, in that, only drug particles on the exposed surface (exterior surface or pore walls) of the coating detach from the stent and into the body lumen.

Thus, a need exists for a method and apparatus for more controllable drug elution within a body lumen.

### SUMMARY OF THE INVENTION

An apparatus includes a base and a coating disposed on the base. A therapeutic agent is disposed on at least one of the base or the coating. A vibration device is coupled to the base. The vibration device is configured to cause movement of the base such that at least a portion of the therapeutic agent is released from the base or the coating. A method includes inserting a stent into a body lumen of a patient. The stent has a base, a coating disposed on at least a portion of the base, and a therapeutic agent carried by at least one of the base or the coating. The stent is vibrated such that at least a portion of the therapeutic agent is released from the stent.

### BRIEF DESCRIPTION OF THE DRAWINGS

The invention is described with reference to the accompanying drawings. In the drawings, like reference numbers indicate identical or functionally similar components.

FIG. 1 is a schematic illustration of a medical device according to an embodiment of the invention.

FIG. 2 is a side perspective view of a medical device according to an embodiment of the invention.

FIG. 3 is a cross-sectional view taken along line 3-3 in FIG. 2.

FIG. 4 is a cross-sectional view taken along line 4-4 in FIG. 2.

FIG. 5 is a cross-sectional view taken along line 5-5 in FIG. 2.

FIG. 6 is a side perspective view of a medical device according to an embodiment of the invention shown within a body lumen of a patient.

FIG. 7 is a partial cross-sectional side view of a medical device according to an embodiment of the invention.

FIG. 8 is side view of a medical device according to an embodiment of the invention.

FIG. 9 is a cross-sectional view taken along the line 9-9 in FIG. 8.

FIG. 10 is a flowchart illustrating a method for using a medical device according to an embodiment of the invention.

### DETAILED DESCRIPTION

Oscillation technology can be integrated with drug carrying stents to provide new procedures for controlling the amount and timing of drug elution from a stent. Such controlled drug elution can be, for example, customized for the particular patient and medical need.

In one embodiment, an apparatus includes a base and a coating disposed on the base. A therapeutic agent is disposed on at least one of the base or the coating. A vibration device is coupled to the base. The vibration device is configured to cause movement of the base such that at least a portion of the therapeutic agent is released from the base or the coating. A method includes inserting a stent into a body lumen of a patient. The stent has a base, a coating disposed on at least a portion of the base, and a therapeutic agent carried by at least one of the base or the coating. The stent is vibrated such that at least a portion of the therapeutic agent is released from the stent.

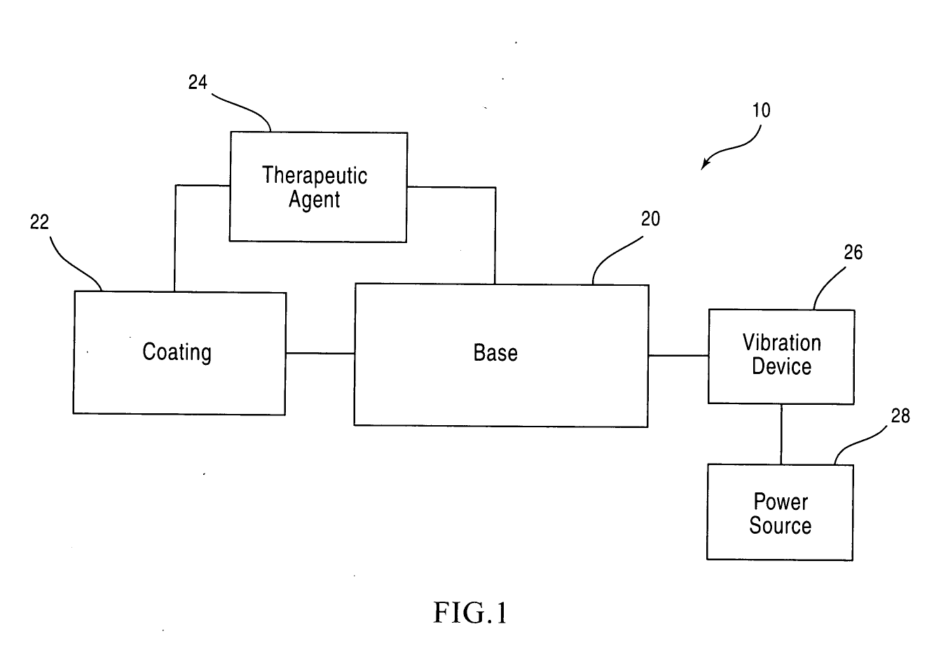

FIG. 1 is a schematic illustration of a medical device according to an embodiment of the invention. A medical device 10 includes a base 20, and a coating 22 disposed on the base 20. A therapeutic agent 24 can be disposed on or embedded within the base 20 and/or the coating 22. A vibration device 26 can be coupled to the base 20 and can also be coupled to a power source 28.

The base 20 can be, for example, a stent that can be implanted into a body lumen of a patient. The base 20 includes an outer surface and an inner surface, and defines a lumen (not shown in FIG. 1). The base 20 can be a variety of different shapes, sizes and structural configurations, and can be constructed with a variety of different types of materials. For example, the base 20 can be constructed with biocompatible and/or bioabsorbable metals, and/or plastics. The base 20 can have, for example, a continuous surface, a matrix configuration (e.g., defining openings on the walls of the stent that are in communication with the lumen), a spiral configuration, or any other suitable stent construction.

The base 20 can be formed, for example, from bioabsorbable and/or biocompatible materials, including metals, polymers, and bioactive glass. Suitable bioabsorbable metals can include, for example, known magnesium alloys, including formulations such as the magnesium alloy disclosed in U.S. Patent Application No. 2002/0004060 (the disclosure of which is incorporated herein by reference in its entirety), which includes approximately 50-98% magnesium, 0-40% lithium, 0-5% iron and less than 5% other metals. Other suitable formulations include a magnesium alloy having greater than 90% magnesium, 3.7%-5.5% yttrium, and 1.5%-4.4% rare earths, as disclosed in U.S. Patent Application No. 2004/0098108 (the disclosure of which is incorporated herein by reference in its entirety). polyethylene terephthalate, polytetrafuoroethylene, polyacetal, poly-L-lactide, polyactic acid, polyanhydrides, polyorthoesters, polyphosphate esters, poly iminocarbonates, polyurethane, polyhydroxy butyrates, polycaprolactones, polytrimethylene carbonates. Alternatively, the base 20 can be formed of a suitable polymer material, such as, for example, polyethylene terephthalate, polytetrafuoroethylene, polyacetal, poly-L-lactide, polyanhydrides, polyorthoesters, polyphosphate esters, poly iminocarbonates, polyurethane, polyhydroxy butyrates, polytrimethylene carbonates, polyactic acid, polyglycolic acid, collagen, polycaprolactone, hylauric acid, adhesive protein, co-polymers of these materials, as well as composites and combinations thereof. Known bioabsorbable polymer stents, which also have drug eluding capabilities include the stents disclosed in U.S. Pat. Nos. 5,464,450; 6,387,124; and 5,500,013 (the disclosures of which are incorporated herein by reference in their entirety).

The coating 22 can be disposed on the base 20 in a variety of configurations. For example, the coating 22 can be disposed on the inner surface of the base 20, the outer surface of the base 20 or both. The coating 22 can be disposed on a portion of the base 20 or substantially covering the inner surface of the base 20, the outer surface of the base 20 and/or both.

The coating 22 can also be formed of a variety of bioabsorbable and/or biocompatible materials. The material may or may not be fully bioabsorbable, depending on whether it is desired to have some or all of the coating 22 remain in the body lumen. The coating 22 can be constructed, for example, with a polymer material. Suitable polymers can include those bioabsorbable polymers discussed above for the base 20. A specific example of a bioabsorbable polymer material incorporating a drug is described in U.S. Pat. No. 5,464,450 and includes a poly-L-lactide

As stated above, the therapeutic agent 24 can be disposed on or embedded within the coating 22 and/or the base 20. As used herein, the term “therapeutic agent” includes, but is not limited to, one or more of any therapeutic agent and/or active material, such as drugs, genetic materials, and biological materials. Suitable genetic materials include, but are not limited to, deoxyribonucleic acid (DNA) or ribonucleic acid (RNA), such as, without limitation, DNA/RNA encoding a useful protein, DNA/RNA intended to be inserted into a human body including viral vectors and non-viral vectors, and RNAi (RNA interfering sequences). Suitable viral vectors include, for example, adenoviruses, gutted adenoviruses, adeno-associated viruses, retroviruses, alpha viruses (Semliki Forest, Sindbis, etc.), lentiviruses, herpes simplex viruses, ex vivo modified and unmodified cells (e.g., stem cells, fibroblasts, myoblasts, satellite cells, pericytes, cardiomyocytes, skeletal myocytes, macrophage), replication competent viruses (e.g., ONYX-015), and hybrid vectors. Suitable non-viral vectors include, for example, artificial chromosomes and mini-chromosomes, plasmid DNA vectors (e.g., pCOR), cationic polymers (e.g., polyethyleneimine, polyethyleneimine (PEI)) graft copolymers (e.g., polyether-PEI and polyethylene oxide-PEI), neutral polymers PVP, SP1017 (SUPRATEK), lipids or lipoplexes, nanoparticles and microparticles with and without targeting sequences such as the protein transduction domain (PTD).

Suitable biological materials include, but are not limited to, cells, yeasts, bacteria, proteins, peptides, cytokines, and hormones. Examples of suitable peptides and proteins include growth factors (e.g., FGF, FGF-1, FGF-2, VEGF, Endothelial Mitogenic Growth Factors, and epidermal growth factors, transforming growth factor α and β, platelet derived endothelial growth factor, platelet derived growth factor, tumor necrosis factor α, hepatocyte growth factor and insulin-like growth factor), transcription factors, proteinkinases, CDK inhibitors, thymidine kinase, and bone morphogenic proteins (BMP's), such as BMP-2, BMP-3, BMP-4, BMP-5, BMP-6 (Vgr-1), BMP-7 (OP-1), BMP-8. BMP-9, BMP-10, BMP-11, BMP-12, BMP-13, BMP-14, BMP-15, and BMP-16. Currently preferred BMP's are BMP-2, BMP-3, BMP-4, BMP-5, BMP-6, and BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at a desired site. The delivery media can be formulated as needed to maintain cell function and viability. Cells include, for example, whole bone marrow, bone marrow derived mono-nuclear cells, progenitor cells (e.g., endothelial progentitor cells), stem cells (e.g., mesenchymal, hematopoietic, neuronal), pluripotent stem cells, fibroblasts, macrophage, and satellite cells.

The term “therapeutic agent” and similar terms also can include non-genetic agents, such as: anti-thrombogenic agents such as heparin, heparin derivatives, urokinase, and PPack (dextrophenylalanine proline arginine chloromethylketone); anti-proliferative agents such as enoxaprin, angiopeptin, or monoclonal antibodies capable of blocking smooth muscle cell proliferation, hirudin, and acetylsalicylic acid, amlodipine and doxazosin; anti-inflammatory agents such as glucocorticoids, betamethasone, dexamethasone, prednisolone, corticosterone, budesonide, estrogen, sulfasalazine, and mesalamine; antineoplastic/antiproliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, taxol and its analogs or derivatives; anesthetic agents such as lidocaine, bupivacaine, and ropivacaine; anti-coagulants such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors and tick antiplatelet peptides; vascular cell growth promotors such as growth factors, Vascular Endothelial Growth Factors (VEGF, all types including VEGF-2), growth factor receptors, transcriptional activators, Insulin Growth Factor (IGF), Hepatocyte Growth Factor (HGF), and translational promotors; vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents, vasodilating agents, and agents which interfere with endogenous vasoactive mechanisms; anti-oxidants, such as probucol; antibiotic agents, such as penicillin, cefoxitin, oxacillin, tobranycin; angiogenic substances, such as acidic and basic fibrobrast growth factors, estrogen including estradiol (E2), estriol (E3) and 17-Beta Estradiol; and drugs for heart failure, such as digoxin, beta-blockers, angiotensin-converting enzyme (ACE) inhibitors including captopril and enalopril.

Some therapeutic materials include anti-proliferative drugs such as steroids, vitamins, and restenosis-inhibiting agents such as cladribine. Restenosis-inhibiting agents include microtubule stabilizing agents such as Taxol, paclitaxel, paclitaxel analogues, derivatives, and mixtures thereof. For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, and 2′-O-ester with N-(dimethylaminoethyl) glutamide hydrochloride salt. Other therapeutic materials include nitroglycerin, nitrous oxides, antibiotics, aspirins, digitalis, and glycosides.

As stated above, the medical device 10 includes a vibration device 26. The vibration device 26 can be, for example, an oscillator, such as a micro-oscillator, that is coupled to the base 20. The vibration device 26 is coupled to the base 20 such that collectively the vibration device 26 and the base 20 can be disposed within a body lumen of a patient. In some embodiments, the vibration device 26 can include multiple oscillators disposed on the base 20 at selected locations on the base 20.

The vibration device 26 can vibrate the base 20 at a resonance frequency associated with the particular configuration of medical device 10. For example, each medical device 10 configuration can have a set of associated resonance frequencies. The resonance frequencies can vary depending on the configuration of those portions of the medical device 10 that are configured to be disposed within a body lumen of a patient. Those portions can include, for example, the base 10, the coating 22, and vibration device. For example, the material composition and structure of base 20, the amount and location of the coating 22, the amount and location of the therapeutic agent 24, and the size and location of the vibration device 26 can all affect the resulting set of resonance frequencies for a medical device 10. Vibration device 26 can be activated at a frequency from the set of resonance frequencies so that the base 20, coating 22, and vibration device 26 collectively vibrate without causing damage to the medical device 10. In some embodiments, the portions of medical device 10 that are configured to be disposed within the body lumen of a patient (e.g., the base 20, coating 22, vibrating device 26) are characterized before insertion. In other words, the set of resonance frequencies associated with the portions of the medical device 10 to be inserted are determined. From this set of resonance frequencies, one or more resonance frequencies that vibrate without damaging the medical device 10 are selected. This process of identifying a set of resonance frequencies that vibrate without damaging the medical device 10 is described further in U.S. Pat. No. 6,729,336.

Upon activation, the vibration device 26 can vibrate the base 20 such that the therapeutic agent 24 is released from either the base 20 or coating 22, or both (depending on the particular configuration of medical device 10) at a rate different from a rate of release associated with the therapeutic agent 24 without the base 20 being vibrated. For example, the therapeutic agent 24 can be naturally released from the medical device 10 as a function of time and/or temperature changes based on the particular material composition of the therapeutic agent 24, the base 20 and/or the coating 22 on which the therapeutic agent is disposed. The vibration of the base 20, can cause the natural release of the therapeutic agent to be modified. For example, in some situations, the release of the therapeutic agent 24 occurs faster than it would naturally be released. In other situations, a greater amount of the therapeutic agent 24 can be released. Thus, the medical device 10 can be configured to achieve a desired release amount and timing of release of the therapeutic agent 24. Although the vibration device 26 is described here as vibrating the base 20, it is to be understood that the base 20 is vibrated collectively with the other portions of the medical device 10 configured to be inserted into a body lumen of a patient. The reference to the base 20 is for simplicity only.

An oscillator of vibration device 26 can be one or more of a variety of different configurations. For example, oscillation technologies, and particularly micro-oscillation technologies, are used in the micro-electro-mechanical systems (MEMS) industry in semiconductors, wireless devices and the like. Oscillation technology can oscillate at tunable Q factors (10-10,000), amplitudes, frequencies, wave forms, etc., to set up oscillations that are sensitive to the tunable parameters. Specific materials suited for tunable oscillators include, for example, quartz and silicon composites. Oscillation technology can be incorporated in cardiovascular stent applications as disclosed in U.S. Pat. No. 6,729,336, the disclosure of which is hereby incorporated by reference in its entirety.

Oscillators can be attached to the base 20 by a variety of different micro-mechanical methods. For example, known attachment methods conducive to lower profile, high shock resistance, and/or high frequency stability include methods based on Graded Glass, which is used in bulb manufacturing and is commonly used to join quartz to tungsten or molybdenum; Cup Seal, which involves attaching quartz through for example, flame torching, to a thin-walled metal cup under vacuum, followed by a brazing procedure; Ribbon Seal, which is similar to the Cup Seal method and involves attaching quartz to a thin-walled ribbon under vacuum; and Swage, which is known for use in attaching radio opaque (RO) markers to stents. Such RO markers can be constructed, for example, with a platinum, gold, or tantulum material with a thin-walled tubular configuration. Other possible methods for attaching an oscillator to the base 20 include methods using solder seal, all quartz, and various welding methods such as, resistance weld, seam weld, epoxy weld; and cold weld techniques.

As stated above, in some embodiments, the vibration device 26 can also be coupled to a power source 28. The power source 28 can provide, for example, electrical energy to the vibration device 26. In some embodiments, the power source 28 is co-located with the vibration device 26. In other embodiments, the power source 28 is remotely located from the vibration device 26, for example, the vibration device 26 can include a wire (not shown in FIG. 1) coupled to an oscillator (not shown in FIG. 1). In such an embodiment, the wire can extend through a lumen of a catheter and be coupled to the power source 28. In other embodiments, the vibration device 26 is activated via a wireless power source 28. The power source 28 can apply energy by remotely sending a signal, such as a radio frequency signal, an optical signal, or an electrical signal, to the vibration device 26 to cause the vibration device 26 to vibrate or oscillate.

In some embodiments, the medical device 10 also includes a balloon (not shown in FIG. 1) that can be removably coupled to the base 20. In alternative embodiments, the balloon is fixedly coupled to the base 20. In an embodiment of medical device 10 that includes a balloon, the balloon can have a collapsed configuration and an expanded configuration. The balloon, in the collapsed configuration, can be inserted into the lumen of the base 20. The balloon can be moved to the expanded configuration after being inserted within the lumen of the base 20. In the expanded configuration, the balloon can have an outer diameter slightly smaller than an inner diameter of the base 20. The balloon in the collapsed configuration can be inserted into the lumen of the base 20 either after the base 20 has been inserted into the body lumen of the patient or prior to inserting the base 20 into the body lumen. In such an embodiment including a balloon, the vibration device 26 can be coupled to the balloon. For example, an oscillator can be coupled to the balloon and be activated as described above. Because of the close proximity of the balloon to the base 20, as the vibration device 26 is activated, the base 20 and other portions of the medical device 10 that are configured to be inserted into a body lumen will collectively vibrate.

The vibration device 26 can be activated causing the base 20 to vibrate upon insertion and/or at a later time depending on the desired timing and amount of release of the therapeutic agent 24. As the base 20 vibrates, at least a portion of the therapeutic agent 24 is released from either the base 20, the coating 22, or both, depending on the particular embodiment of medical device 10. In another use, the vibration device 26 can be activated prior to insertion of the base 20 into the body lumen. This procedure may be desired, for example, to remove an initial portion of the therapeutic agent 24 prior to implanting the base 20. The medical device 10 can also be used to measure restenosis of the body lumen in conjunction with elution of the therapeutic agent 24. For example, prior to vibrating the base 20, restenosis of the body lumen can be measured using techniques described in U.S. Pat. No. 6,729,336. The base 20 can then be vibrated at a level, and for a time period based on the measured restenosis.

The various configurations of medical device 10 are such that the amount of release of the therapeutic agent 24 can be controlled and customized for the particular patient. A number of factors can affect the amount of release of the therapeutic agent 24, such as the particular frequency signal used to activate the vibration device 26, the amplitude of the oscillator when the vibration device 26 is activated, the amount of therapeutic agent 24 carried on the base 20 (or coating 22), and the amount and formulation of the coating 22 when the therapeutic agent is contained within the coating 22. The therapeutic agent 24 can be released on demand by activating the vibration device 26. In some embodiments, the vibration can commence upon sensing by an electroencephalogram (EEG) monitoring device, such as a pacing or defibrillation implant, sensing a condition of a patient. For example, cardiac electrical transmissions or blood chemistry abnormalities can be detected and cause an activation signal to start the oscillation/vibration, thereby releasing more therapeutic agent into the affected vessel.

Several exemplary embodiments of a medical device are now described. These embodiments are only examples, and many other combinations and formulations of base 20, coating 22, vibration device 26 and therapeutic agent 24 are possible. In addition, any of the above-described features and functions of medical device 10, as well as methods of use of medical device 10, are applicable to the embodiments of a medical device and methods of use described below.

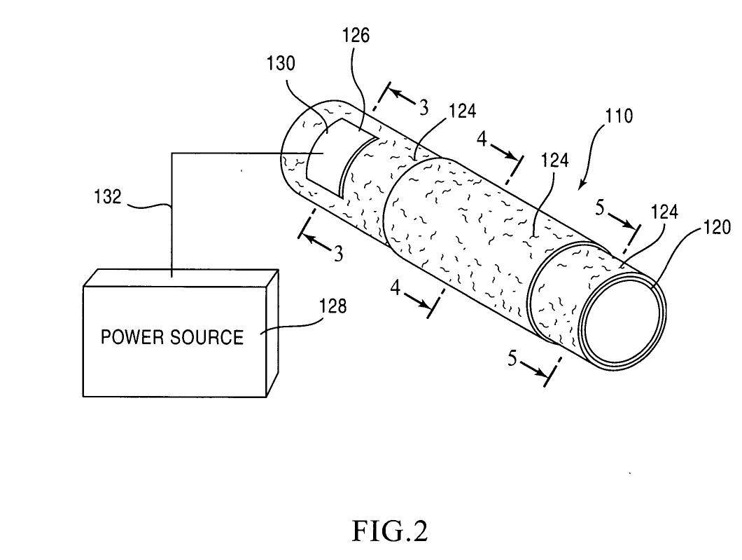

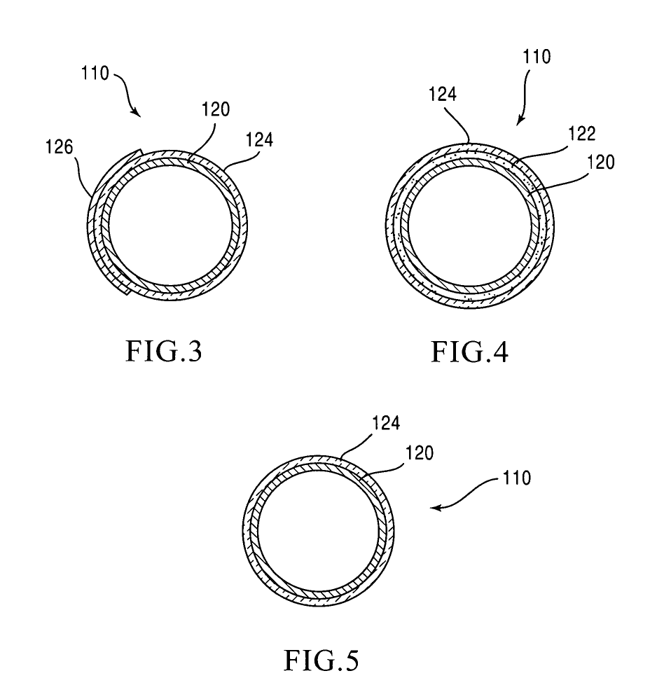

FIGS. 2-5 illustrate a medical device according to another embodiment of the invention. Medical device 110 includes a base 120 and a coating 122 disposed on an outer surface of the base 120. A therapeutic agent 124 can be carried by either the base 120, the coating 122 or both. In this embodiment, the therapeutic agent 124 is disposed on a portion of both the base 120 and the coating 122. A vibration device 126 is also disposed on the base 120. FIG. 3 is a cross-sectional view illustrating the therapeutic agent 124 disposed on a first portion of the base 120; FIG. 4 is a cross-sectional view illustrating the therapeutic agent 124 disposed on the coating 122; and FIG. 5 is a cross-sectional view illustrating the therapeutic agent 124 disposed on a second portion of the base 120. Although the therapeutic agent 124 is shown as a layer of material disposed on the base 120 and coating 122, the therapeutic agent 124 can alternatively be embedded or incorporated within the base 120 and/or coating 122. The vibration device 126 includes an oscillator 130 coupled to the base 120, and a wire 132 coupling the oscillator 130 to a power source 128. In this embodiment, the vibration device 126 is activated via the power source 128, and the power supplied by the power source 128 travels through the wire 132 to the oscillator 130.

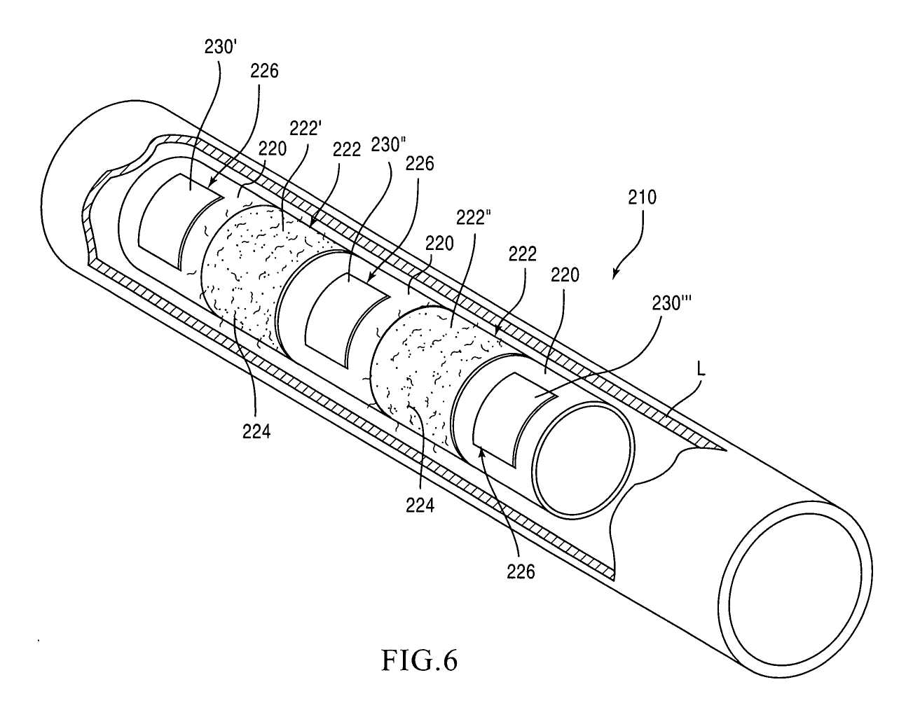

FIG. 6 illustrates a medical device according to another embodiment of the invention shown implanted within a body lumen L shown in partial-cutaway. A medical device 210 includes a base 220 and a coating 222 disposed on an outer surface of the base 220. In this embodiment, the coating 222 includes a first portion 222′ and a second portion 222″. A therapeutic agent 224 is carried by the coating 222. A vibration device 226 is disposed on the base 220 and includes a oscillators 230′, 230″, and 230′″. The oscillators 230′, 230″, and 230′″ can be activated via a wireless signal from a power source (not shown in FIG. 6) at a location external to the body lumen L. When the vibration device 226 is activated, the oscillators 230′, 230″, and 230′″ cause the base 220 to vibrate and at least a portion of the therapeutic agent 224 is released from the coating 222 and into the body lumen L.

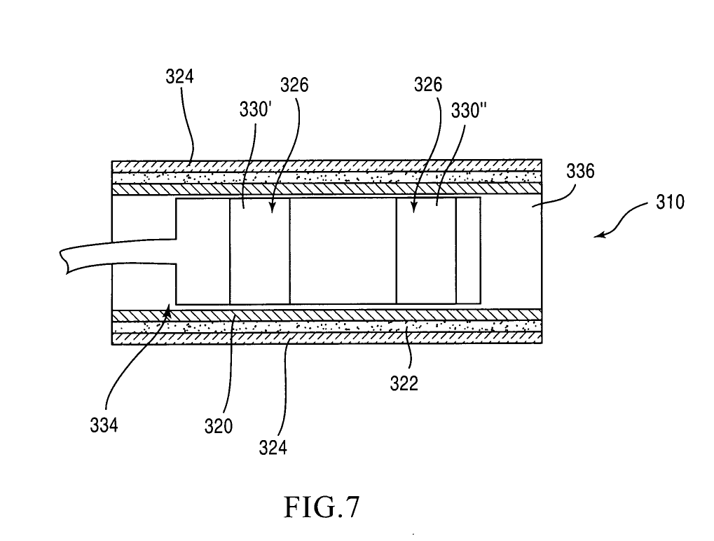

FIG. 7 illustrates a medical device according to another embodiment of the invention shown partially in cross-section. A medical device 310 includes a base 320, and a coating 322 disposed on the base 320. The coating 322 substantially covers an outer surface of the base 320. A therapeutic agent 324 can be carried by the coating 322 or disposed as a layer on coating 322 as shown in FIG. 7. An expandable device, such as a balloon 334 is disposed in a lumen 336 of the base 320. As shown in FIG. 7, the balloon 334 is in an expanded configuration. A vibration device 326 is disposed on an outer surface of the balloon 334. The vibration device 326 includes a first oscillator 330′ and a second oscillator 330″.

The balloon 334 in the collapsed configuration can be inserted into the lumen 336 of the base 320. The balloon 334 can be expanded to the expanded configuration in which an outer diameter of the balloon 334 is slightly smaller than an inner diameter of the lumen 336 of the base 320. As with the previous embodiment, the vibration device 326 (i.e., oscillators 330′ and 330″) can be activated remotely via energy applied from an external power source (not shown in FIG. 7). When the oscillators 330′ and 330″ are activated, the balloon 334 will vibrate, which in turn causes the base 320 to vibrate. The vibration motion of the base 320 causes the therapeutic agent 324 to be released from the coating 322.

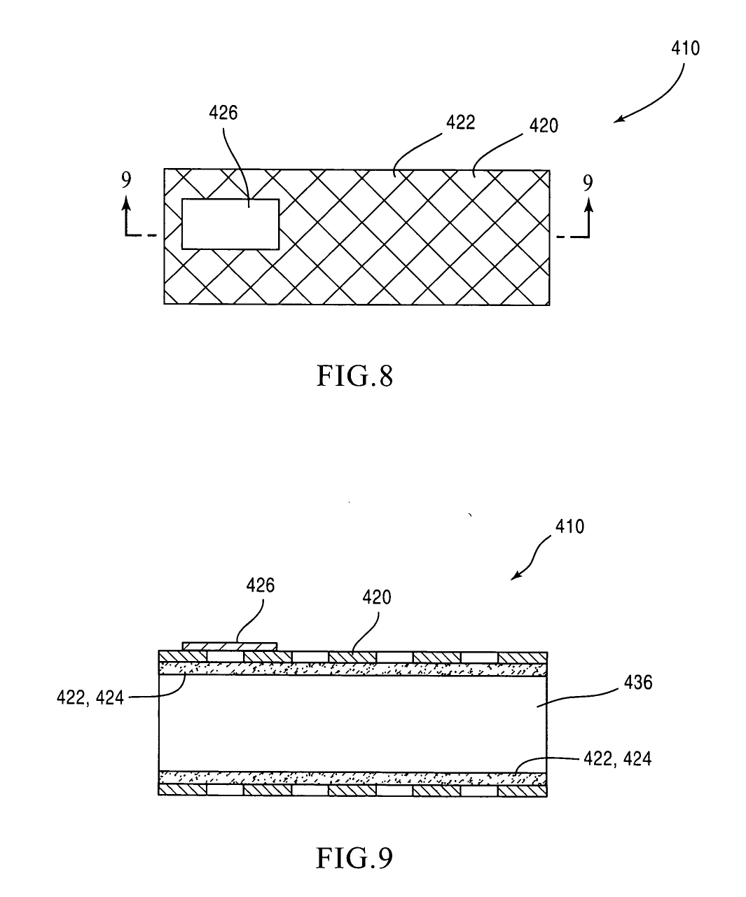

FIG. 8 is a side view, and FIG. 9 is a cross-sectional view of a medical device according to another embodiment of the invention. A medical device 410 includes a base 420 defining a lumen 436. The medical device 410 is constructed with an open or lattice type configuration. A coating 422 is disposed on an inner surface of the base 420, as shown in FIG. 9. The coating 422 can be, for example, a bioabsorbable material that naturally bioabsorbs into the body over time. A vibration device 426 is coupled to an outer surface of the base 420. A therapeutic agent 424 is disposed on the coating 422, such that when the medical device 420 is vibrated by activating the vibrating device 426, at least a portion of the therapeutic agent is released from the coating 422. The lattice configuration of the base 420 allows the therapeutic agent 422 to pass through the walls of the base 420 and into contact with the inner walls of the body lumen. A portion of the therapeutic agent 424 can also be released into the lumen 436 and carried into the body lumen through fluid flow through the body lumen (e.g., blood flow).

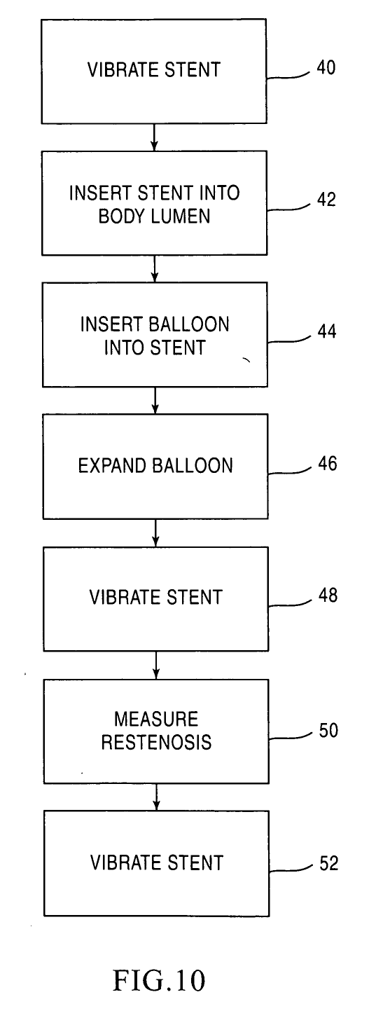

A method of using a medical device is now described with reference to the flowchart illustrated in FIG. 10. At step 40, a stent (e.g., base 20, 120, 220, 320, 420) can be vibrated as described above, prior to implanting the stent into a body lumen of a patient. Step 40 is optional, as it is not required that the stent be vibrated prior to insertion into a body lumen. The stent includes a coating disposed thereon, and when the stent is vibrated at least a portion of a therapeutic agent carried by the coating, the stent, or both, will be released. The stent is implanted into a body lumen of a patient at step 42.

In some embodiments, an expandable member, such as a balloon can be inserted in a collapsed configuration into a lumen of the stent at step 44, either before or after the stent has been implanted into the body lumen of the patient. The balloon can then be expanded to an expanded configuration at step 46, while disposed within the lumen of the stent. After the stent has been implanted, the stent can be vibrated at step 48, such that at least a portion of the therapeutic agent is released from the stent, the coating, or both, and into the body lumen of the patient. The vibrating can be done immediately upon implantation and/or at a later time period. Step 48 is optional in that a stent does not have to be vibrated after implanting the stent into the body lumen. In such a situation, the stent may be vibrated prior to installation to release an initial amount of therapeutic agent and then allowed to naturally release the therapeutic agent after implantation into the body lumen.

In an embodiment including a balloon, the stent can be vibrated by vibrating the balloon, at step 48. For example, the balloon can be vibrated causing vibration of the stent prior to implanting the stent into the body lumen. Alternatively, the balloon can be vibrated after the stent has been implanted into the body lumen.

In some embodiments, restenosis of the body lumen can be measured using the techniques described in U.S. Pat. No. 6,729,336, after the stent has been inserted into the body lumen at step 50. The stent can then optionally be vibrated at step 52 at a level and for a time period determined based on the restenosis measured at step 50.

### Conclusion

While various embodiments of the invention have been described above, it should be understood that they have been presented by way of example only, and not limitation. Thus, the breadth and scope of the invention should not be limited by any of the above-described embodiments, but should be defined only in accordance with the following claims and their equivalents. While the invention has been particularly shown and described with reference to specific embodiments thereof, it will be understood that various changes in form and details may be made.

What is claimed is:

1. An apparatus, comprising:
a base;
a coating disposed on the base;
a therapeutic agent disposed on at least one of the base or the coating; and
a vibration device coupled to the base, the vibration device configured to cause movement of the base such that at least a portion of the therapeutic agent is released from the at least one of the base or the coating.

2. The apparatus of claim 1, wherein the vibration device is configured to vibrate the base at a resonance frequency associated with the base.

3. The apparatus of claim 1, wherein the vibration device includes a plurality of oscillators disposed at selected locations on the base such that, upon activation of the vibration device, the base vibrates at a resonance frequency associated with the base.

4. The apparatus of claim 1, wherein the vibration device is configured to vibrate the base such that the therapeutic agent is released from the at least one of the base or coating at a rate different from a rate of release associated with the therapeutic agent without being vibrated.

5. The apparatus of claim 1, wherein the base and the vibration device are dimensioned such that the base and the vibration device collectively can be disposed within a body lumen of a patient, the vibration device being configured to be remotely activated.

6. The apparatus of claim 1, wherein the vibration device includes a wire coupled to a power source.

7. A method, comprising:
inserting a stent into a body lumen of a patient, the stent having a base, a coating disposed on at least a portion of the base, and a therapeutic agent carried by at least one of the base or the coating; and
vibrating the stent such that at least a portion of the therapeutic agent is released from the stent.

8. The method of claim 7, wherein the vibrating is prior to the inserting the stent into the body lumen of the patient.

9. The method of claim 7, wherein the vibrating is after the inserting the stent into the body lumen of the patient.

10. The method of claim 7, wherein the stent further includes a vibration device coupled to the base, the method further comprising applying energy to the vibration device, the stent vibrating in response to the applying.

11. The method of claim 7, further comprising:
inserting a balloon in a collapsed configuration into a lumen of the stent, a vibration device being coupled to the balloon; and
activating the vibration device, the stent vibrating in response to the activating.

12. The method of claim 7, wherein the stent further includes a vibration device coupled to the base, the vibration device being coupled to a power source, and the vibrating includes sending a signal remotely from the power source to the vibration device.

13. The method of claim 7, wherein the apparatus further includes a vibration device coupled to the base, the vibration device includes an oscillator, a wire coupled to the oscillator and a power source; and
the vibrating includes applying energy to the oscillator remotely from the power source.

14. The method of claim 7, wherein the vibrating includes vibrating the stent at a resonance frequency associated with the base.

15. The method of claim 7, further comprising:
measuring remotely restenosis of the body lumen prior to the vibrating the stent; and
the vibrating including vibrating remotely the stent for a time period based on the measure of restenosis after the inserting the stent into the body lumen.

16. A method, comprising:
inserting a stent into a body lumen of a patient, the stent having a base, a coating, and a therapeutic agent;
disposing a balloon in a collapsed configuration into a lumen of the stent in a collapsed configuration, the balloon coupled to a vibration device;
expanding the balloon while disposed within the lumen of the stent; and
activating the vibration device such that the stent vibrates at a resonance frequency associated with the base and such that at least a portion of the therapeutic agent is released from the stent and into the body lumen of the patient.

17. The method of claim 16, wherein the vibration device includes an oscillator coupled to the balloon, and
the activating includes remotely sending an activation signal to the oscillator.

18. The method of claim 16, wherein the vibration device includes an oscillator coupled to the balloon, a wire coupled to the oscillator, and a power source, and
the activating includes sending an activation signal from the power source to the oscillator via the wire.

19. The method of claim 16, further comprising:
activating the vibration device prior to the inserting such that at least a portion of the therapeutic agent is released from the stent.

20. An apparatus, comprising:
a base;
a coating disposed on the base;
a therapeutic agent disposed on at least one of the base or the coating;
a balloon removably coupled to the base; and
a vibration device coupled to the balloon, the vibration device configured to cause movement of the base such that at least a portion of the therapeutic agent is released from the at least one of the base or the coating.

21. The apparatus of claim 20, wherein the vibration device is configured to vibrate the base at a resonance frequency associated with the base.

22. The apparatus of claim 20, wherein the vibration device includes a plurality of oscillators disposed at selected locations on the balloon such that, upon activation of the vibration device, the base vibrates at a resonance frequency associated with the base.

23. The apparatus of claim 20, wherein the vibration device is configured to vibrate the base such that the therapeutic agent is released from the at least one of the base or the coating at a rate different from a rate of release associated with the therapeutic agent without being vibrated.

24. The apparatus of claim 20, wherein the base and the vibration device are dimensioned such that the base and the vibration device collectively can be disposed within a body lumen of a patient, the vibration device being configured to be remotely activated.
